(19) United States
(12) Patent Application Publication
(10) Pub. No.: US 2025/0170085 A1
(43) Pub. Date: May 29, 2025

(54) TOPICAL FORMULATION TO DECREASE SKIN INFLAMMATION OR REDNESS

(71) Applicant: University of Pittsburgh - Of the Commonwealth System of Higher Education, Pittsburgh, PA (US)

(72) Inventor: Daniel H. Kaplan, Pittsburgh, PA (US)

(73) Assignee: University of Pittsburgh - Of the Commonwealth System of Higher Education, Pittsburgh, PA (US)

(21) Appl. No.: 18/843,152

(22) PCT Filed: Mar. 2, 2023
(86) PCT No.: PCT/US2023/014383

§ 371 Date: Aug. 30, 2024

Related U.S. Application Data
(60) Provisional application No. 63/316,158, filed on Mar. 3, 2022.

(51) Int. Cl. A61K 31/198; A61K 8/34; A61K 8/44; A61K 9/00; A61K 31/045; A61K 45/06; A61P 17/04; A61Q 5/02; A61Q 19/10

(52) U.S. Cl. CPC A61K 31/198; A61K 8/34; A61K 8/44; A61K 9/0014; A61K 31/045; A61K 45/06; A61P 17/04; A61Q 5/02; A61Q 19/10

(57) ABSTRACT

Topical compositions including β-alanine and a transient receptor potential cation channel subfamily M (melastatin) member 8 (TRPM8) agonist, such as menthol, are disclosed. Also disclosed are methods of decreasing skin inflammation or redness in a subject, and methods of decreasing frequency or duration of symptoms of inflammatory skin conditions, such as rosacea.

### CROSS REFERENCE TO RELATED APPLICATION

This claims the benefit of U.S. Provisional Application No. 63/316,158, filed Mar. 3, 2022, which is incorporated herein by reference.

### FIELD

This disclosure relates to cosmetics, specifically to topical compositions and their use to decrease skin inflammation or redness.

### BACKGROUND

Dermal mast cells are skin-resident immune cells that quickly respond to allergens by releasing granules containing inflammatory meditators (such as histamine and serotonin). While mast cells play an important role in both adaptive and innate immunity, a number of inflammatory skin conditions implicate mast cell activation, including rosacea, urticaria, mastocytosis, allergy, dermatitis, acne, psoriasis, and scleroderma. Mast cell activation is also considered to be the etiology of other skin conditions, such as chronic flushing (e.g., alcohol flush, or due to emotional response) and is involved with wound healing. Inflammation and/or redness of the skin, particularly of facial skin, can be embarrassing and painful for affected individuals. Inflammatory skin conditions are also often chronic conditions that have no cure, requiring long term management. Thus, topical compositions suitable for daily use that can decrease redness or inflammation are desired.

### SUMMARY

Disclosed herein are topical compositions that include an effective amount of β-alanine and an effective amount of a transient receptor potential cation channel subfamily M (melastatin) member 8 (TRPM8) agonist. In some implementations, the topical composition includes about 0.2% to about 15% weight by weight (w/w) β-alanine, for example, about 1% to about 10% w/w 1-alanine, or about 2.5% to about 7.5% w/w β-alanine. In some examples, the effective amount is about 5% w/w β-alanine. In some implementations, the TRPM8 agonist is menthol. In some implementations, the topical composition includes about 0.1% to about 5% w/w menthol, for example, about 0.1% to about 2% w/w menthol, or about 0.5% to about 1.5% w/w menthol. In some examples, the topical composition includes about 0.5% w/w menthol. The topical composition can include additional agents, for example, a topical UV protectant, antibiotic, steroid, non-steroidal anti-inflammatory agent, calcineurin inhibitor, JAK inhibitor, α2-adrenergic receptor agonist, α1A-adrenoceptor agonist, or combinations thereof. In some implementations, the topical composition does not comprise an anesthetic or topical steroid. The topical composition can be formulated in any suitable form for topical application, such as an ointment, cream, gel, lotion, shampoo, or soap. In some examples, the topical composition is formulated as an emollient or moisturizer, and/or is formulated for facial application.

Also disclosed herein are methods of decreasing skin inflammation or redness, which include administering an effective amount of the topical composition described herein to an area of skin of the subject affected by inflammation or redness. In some implementations, the area of skin is affected by an inflammatory skin condition, chronic flushing, or a wound. In some implementations, the method includes selecting a subject with an inflammatory skin condition, chronic flushing, or a wound. Also disclosed are methods of decreasing frequency, duration, or severity of rosacea symptoms in a subject. The method includes selecting a subject with rosacea and administering the topical composition described herein to an area of skin affected by rosacea. In some examples, the subject has erythematotelangiectatic rosacea, papulopustular rosacea, and/or phymatous rosacea. In some implementations, the topical composition is administered repeatedly, for example, chronically over days, weeks, months, or even the remaining lifetime of the subject. In some implementations, the topical composition is administered several times a week (e.g., about 3, 4, 5, or 6 times a week), daily, or twice daily. In some examples, the method further includes administering a topical UV protectant, antibiotic, steroid, non-steroidal anti-inflammatory agent, calcineurin inhibitor, JAK inhibitor, analgesic, α2-adrenergic receptor agonist, α1A-adrenoceptor agonist, or combinations thereof, before, after, or substantially at the same time as the topical composition.

The foregoing and other objects, features, and advantages of the invention will become more apparent from the following detailed description, which proceeds with reference to the accompanying figures.

### BRIEF DESCRIPTION OF THE DRAWINGS

FIG. 1 shows subjective patient-reported scoring for itch of patients with facially applied cream containing β-alanine at the indicated concentration. The four β-alanine formulations (0%, 2.5%, 5.0%, and 7.5% w/w β-alanine) were sequentially applied to a different location of each patient's cheek skin by a physician. Subjective scoring for itch (0 (none) to 10 (maximum itch)) was recorded over a 5-minute period. Symbols on the bar graph represent individual patients (n=16). Bar height indicates the average value. Error bars represent +/−SEM. ** P<0.01; **** P<0.0001. P-values were calculated using a nonparametric ANOVA Kruskai-Wallis test.

FIGS. 2A and 2B show subjective patient-reported scoring for itch (FIG. 2A) and overall discomfort (FIG. 2B) of facially applied cream containing 5% w/w β-alanine and the indicated concentration of menthol. Each of the four formulations (0%, 0.5%, 1.0%, and 1.5% w/w menthol with 5% w/w f-alanine) were sequentially applied to different locations of a patient's cheek skin by a physician. Scoring was on a scale of 0 to 10, with 0 being no itch or discomfort and 10 being maximum itch or discomfort. Each symbol represents an individual patient (n=10). Bar height indicates the average value. Error bars represent +/−SEM. * P<0.05; ** P<0.01. P-values were calculated using a nonparametric ANOVA Kruskai-Wallis test.

### DETAILED DESCRIPTION

β-alanine can suppress mast cell activation, however, topical use of β-alanine is limited due to the onset of pruritus (itching) following application. Pruritus triggered by topical β-alanine cream is transient, thus, combination with an agent that is able to mask β-alanine induced pruritus for a short period of time could improve tolerability of topical β-alanine compositions.

While many compounds reportedly “suppress itch” when topically applied, most are unsuitable for chronic use and carry the risk of undesirable side-effects. For example, lidocaine and other related -caine medications, which are primarily used as anesthetics, can result in unpleasant numbing of the skin that persists much longer than the approximately 3 minutes required to suppress β-alanine induced itch. In addition, lidocaine and other related -caine medications may also have a slower onset of action than β-alanine induced itch, and thus would be ineffective at reducing itch. Systemic absorption of these mediations also carries risks for CNS and cardiac toxicities, especially in patients with liver disease, and thus are unsuitable for chronic use. Similarly, camphor, which is also primarily used as an anesthetic, can have undesirable side effects following systemic absorption.

Low potency topical steroids may also be used to suppress pruritus, however, the onset of action is likely too slow to provide a benefit in masking β-alanine induced itch. In addition, application of topical steroids to the face can induce acne, worsen rosacea, and carries the risk for the development of striae and steroid addiction, particularly with chronic use. Topical preparation of moisturizers and colloidal oatmeal improves barrier integrity and can decrease pruritus in context in which the barrier is defective, such as atopic dermatitis, however, are unlikely to provide any benefit in masking β-alanine induced pruritus.

The work disclosed here sought to identify a compound capable of masking β-alanine induced itch, but is also well tolerated and suitable for chronic use.

### Terms

Unless otherwise indicated, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B.

Although methods and materials similar or equivalent to those described herein can be used in the practice or testing of the present disclosure, suitable methods and materials are described below. In case of conflict, the present specification, including explanations of terms, will control. In addition, the materials, methods, and examples are illustrative only and not intended to be limiting.

In order to facilitate review of the various implementations of the disclosure, the following explanations of specific terms are provided:

About: unless context clearly indicated otherwise, “about” refers to a range of plus or minus 5% of a reference value. For example, “about 100 grams” refers to 95 grams to 105 grams. In another example, “about 5%” refers to 4.75% to 5.25%.

Acne: an inflammatory disorder of skin that can occur on skin that has sebaceous glands connected to hair follicles. In healthy skin, the sebaceous glands make sebum that empties onto the skin surface through a pore, which is an opening in the follicle. When someone has acne, hair, sebum, and keratinocytes stick together inside the pore, preventing sebum from reaching the surface of the skin and keratinocytes from shedding. The mixture of oil and cells allows bacteria to grow in the plugged follicles, causing inflammation. When the wall of the plugged follicle breaks down, it spills the bacteria, skin cells, and sebum into nearby skin, creating lesions or pimples.

Administration: to provide or give a subject an agent by any effective route. Administration includes topical application.

Agent: any substance or compound that is useful for achieving a particular outcome. For example, the agent can be a topical composition capable of decreasing skin redness or inflammation.

Analgesic: a medication that relieves pain. Non-limiting examples of topical analgesics include capsaicin, diclofenac, lidocaine, methyl salicylate, and trolamine salicylate.

Antibiotic: a compound or substance that kills or substantially slows down the growth of bacteria, fungi, or other microorganisms. An “antibacterial” is a compound or substance that kills or substantially slows the growth of bacteria. An “antifungal” is a compound or substance that kills or substantially slows the growth of fungi.

β-alanine: a non-essential beta amino acid (sometimes also referred to as b-alanine). Beta amino acids are amino acids in which the amino group is attached to the β-carbon (i.e., the carbon two atoms away from the carboxylate group). The IUPAC name for β-alanine is 3-aminopropanoic acid. Purified β-alanine can be obtained commercially, for example, Beyond Raw® Chemistry Labs™ Beta-alanine (GNC Item #369898); or β-alanine (Sigma-Aldrich, #146064).

Control: a reference standard. The control may be any suitable positive or negative control. Several non-limiting examples of controls are provided for illustrative purposes. In one example, the control is a sample or measurement obtained from a subject prior to administering the disclosed topical composition. Another exemplary control is a subject administered a topical composition without βl-alanine, a TRPM8 agonist, or both (empty vehicle), or is not administered any topical composition. In a further example, a suitable control is a historical control or standard reference value or range of values (such as a previously obtained control sample, or group of samples that represent baseline or “normal” values).

A difference between a test sample (e.g., administering the disclosed topical composition to a subject) and a control can be an increase or conversely a decrease. The difference can be a qualitative (e.g., subjective observations) or quantitative (e.g., objective measurements). In some examples, the difference is statistically significant. In some examples, an increase relative to a control is at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%. In some examples, a decrease relative to a control is least about 5%, such as at least about 10%, at least about 20%, at least about 30%, at least about 40%, at least about 50%, at least about 60%, at least about 70%, at least about 80%, at least about 90%, at least about 95%, at least about 98%, or about 100%.

Cosmetically Acceptable Vehicles: many suitable cosmetically acceptable vehicles (carriers) (e.g., pharmaceutically acceptable carriers) have been described. For example, Remington: The Science and Practice of Pharmacy, 23rd Ed. (Academic Press), 2021 (ISBN 9780128200070), describes compositions and formulations suitable for delivery of topical compositions. Use of the term “pharmaceutically acceptable carriers” does not imply that a product is useful only for pharmaceutical purposes. Rather it implies that the product is suitable for administration or consumption by a subject, such as a human. In particular implementations, the vehicle is a carrier for a topical composition, such as a liquid, gel, foam, cream, ointment or lotion. Particularly useful vehicles are those that are cosmetically acceptable for topical applications, such as one or more aqueous systems, glycerin, C₁₋₄ alcohols, fatty alcohols, fatty ethers, fatty esters, polyols, glycols, vegetable oils, mineral oils, liposomes, laminar lipid materials, silicone oils, water, or combinations thereof.

The topical compositions disclosed herein can be formulated in any suitable product form, Such product forms include, but are not limited to, aerosol spray, cream, dispersion, emulsion, foam, gel, liquid, lotion, mousse, ointment, patch, pomade, powder, pump spray, solid, solution, stick, towelette, or the like. The carrier can also be a variety of existing skin lotions, gels, creams, ointments, toners, cleansers, moisturizers or sunscreens to which the disclosed topical composition is added in a desired concentration.

Dermatitis: a general term for skin inflammation. It has many causes and forms. It usually involves itchy, dry skin or a rash. In some cases, it may cause the skin to blister, ooze, crust or flake off. Three common types of dermatitis include atopic dermatitis, seborrheic dermatitis and contact dermatitis. Atopic dermatitis is commonly referred to as eczema. Atopic dermatitis is often the result of a combination of factors, such as dry skin, environmental irritants, stress, hormonal changes, and bacteria on the skin. During flare-ups, patches of skin may appear rough, dry, and itchy. Seborrheic dermatitis (e.g., dandruff or cradle cap) is most common on the scalp, though it can also occur on the face, chest, and around the eyes and ears. It often causes scaly patches, skin discoloration, and dandruff. While the exact cause of seborrheic dermatitis is unknown, stress or lack of sleep can worsen symptoms. Contact dermatitis occurs when a substance causes an adverse inflammatory reaction upon contact with skin. These reactions can develop into rashes that burn, sting, itch, or blister. Contact dermatitis may be the result of an allergic or an irritant reaction. For irritant contact dermatitis an outside substance directly damages the skin and causes a reaction. For allergic contact dermatitis, the outside substance may not directly damage the skin but causes the immune system to react in a way that causes skin damage. Other types of dermatitis include asteatotic dermatitis, diaper dermatitis (diaper rash), dyshidrotic dermatitis, neurodermatitis, nummular dermatitis, perioral/periorificial dermatitis, stasis dermatitis, and dermatitis neglecta.

Dermatographia: an inflammatory skin condition that causes individuals to develop raised welts or a localized hive-like reaction when they scratch their skin. It can also happen when the skin is exposed to pressure or rubbing. This condition is also called skin writing, dermographia, or dermatographic urticaria.

Effective amount: the amount of agent, such as a topical composition, which is sufficient to obtain a desired result, such as decreasing skin redness or inflammation. In some implementations, an effective amount of the disclosed topical composition is an amount that decreases skin redness or inflammation, or suppresses mast cell activation. In some implementations, an effective amount of β-alanine is an amount that decreases skin redness or inflammation. In some examples, the effective amount of β-alanine suppresses mast cell activation. In some implementations, the effective amount of a TRPM8 agonist is an amount that suppresses transient β-alanine induced itch. The effective amount can be administered in a single dose, or in several doses, for example daily. However, the effective amount can depend on the subject, the subject's condition, and the manner of administration.

Excipient: an inactive substance used as a carrier for the active ingredients of a composition. Excipients can include substances that are used to bulk up formulations with active ingredients, allow for convenient and accurate dosage, stabilize the active ingredients, and make the delivery system optically and/or organoleptically acceptable. Examples of cosmetically acceptable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like.

Extract: A solution or other preparation of at least some of the active ingredients of a plant or one or more of its parts, such as its roots, shoots, leave, fruit, or seed. Extracts can be solvent extracts, for example, when plants or plant parts are exposed to a liquid extract solvent (e.g., water, alcohols, acetone, dichloromethane, or chloroform) to remove active ingredients. An extract initially obtained by solvent extraction may be converted into a dried form and still be considered an “extract.”

Increase or Decrease: A positive or negative change, respectively, relative to a control value. An increase is a positive change, such as an increase of at least 25%, at least 50%, at least 75%, at least 100%, at least 200%, at least 300%, at least 400%, or at least 500% as compared to the control value. An increase can be within a range, for example, 25% to 500%, 25% to 400%, 25% to 300%, 25% to 200%, 25% to 100%, 25% to 75%, 25% to 50%, 50% to 500%, 75% to 500%, 100% to 500%, 200% to 500%, 300% to 500%, 400% to 500%, 50% to 100%, 50% to 200%, 50% to 300%, 50% to 400%, 50% to 500%, 100% to 200%, 100% to 300%, 100% to 400%, 100% to 500%, or 250% to 500%.

A decrease is a negative change, such as a decrease of at least 10%, at least 15%, at least 20%, at least 25%, at least 30%, at least 40%, at least 50%, at least 60%, at least 70%, at least 80%, at least 90%, at least 95%, at least 98%, at least 99%, or at least 100% decrease as compared to a control value. A decrease can be within a range, for example, 25% to 100%, 25% to 98%, 25% to 95%, 25% to 90%, 25% to 80%, 25% to 70%, 25% to 50%, 50% to 100%, 75% to 100%, 90% to 100%, 95% to 100%, 98% to 100%, 99% to 100%, 50% to 75%, 50% to 80%, 50% to 90%, 50% to 95%, 50% to 98%, 75% to 80%, 75% to 90%, 75% to 95%, or 75% to 98%.

Inflammation: a localized biological reaction that can produce redness, swelling and pain as a result of damaging stimuli including infection, irritation, injury, or disease. Inflammation can be chronic or acute. Immune cells, such as dermal mast cells, initiate inflammatory responses and release inflammatory mediators (such as histamine, proinflammatory cytokines, or serotonin) responsible for increased blood flow and leakage of plasma fluid into the affected tissue.

Inflammatory skin condition: a skin condition associated with inflammation. Non-limiting examples include rosacea, acute or chronic urticaria, dermatographia, mastocytosis, dermatitis, acne, psoriasis, and scleroderma.

Mast cell: a type of resident inflammatory cell found in the dermal layer of the skin. Mast cell activation helps induce inflammation by quickly releasing a variety of preformed mediators (such as histamine, serotonin, proteases, and proinflammatory cytokines) that are stored within mast cell granules. A number of mechanisms can activate mast cells to induce degranulation, such as IgE during allergic reactions, pathogens (bacteria, viruses, and parasites) or pathogen products, chemicals, neuropeptides, cytokines, or physical stimuli (such as heat or mechanical injury). In addition, prominent mast cell activation is seen at early stages of wound healing.

Mastocytosis: a condition caused by an abnormal accumulation and activation of mast cells in the skin, bone marrow and internal organs. Symptoms of mastocytosis are mainly due to the release of chemicals from the mast cells, and thus produce symptoms similar to an allergic reaction. Flushing due to mast cell-associated histamine release is a common symptom. In some cases, massive chemical release from the mast cells (degranulation) may lead to life-threatening episodes of anaphylaxis (anaphylactic shock). The most common triggers include, but are not limited to, insect stings, physical stress (heat, cold, mechanical irritation of the skin, exercise), emotional stress, alcohol, spicy foods and medications, including aspirin and non-steroidal anti-inflammatory drugs (NSAIDS), narcotics, muscle relaxants, radiocontrast material, among others.

Cutaneous mastocytosis is a type of mastocytosis that affects the skin. Sub-types of cutaneous mastocytosis include maculopapular cutaneous mastocytosis, solitary cutaneous mastocytoma, and diffuse cutaneous mastocytosis. Maculopapular cutaneous mastocytosis is characterized by itchy, brown patches on the skin. The patches may occur on scalp, neck, trunk and extremities. Solitary cutaneous mastocytoma is a localized form of cutaneous mastocytosis. Typically, there is a single lesion, or up to 3 individual lesions, which are usually found early in life and resolve spontaneously with age. Diffuse cutaneous mastocytosis is the most severe form of cutaneous mastocytosis. The skin is diffusely thickened and has a rough texture, generally without individual distinct lesions. Additional symptoms associated with DCM include itching, blistering, decreased blood pressure (hypotension), diarrhea, gastrointestinal bleeding, reddening of the skin (flushing) and anaphylactic shock.

Menthol: a monoterpenoid that can be made synthetically, or obtained from the oils of corn, mint, peppermint, or other mints. The preferred IUPAC name of menthol is 5-methyl-2-(propan-2-yl)cyclohexan-1-ol. Purified Menthol can be obtained commercially, for example, Menthol Essential Oil (Oakland Gardens, 100% pure Mentha arvensis extract); or Menthol (Sigma-Aldrich, #M2772).

Psoriasis: an immune-mediated disease skin that causes visible signs of inflammation, such as raised plaques (plaques may look different for different skin types) and scales on the skin. The red, itchy scaly patches are most commonly found on the knees, elbows, trunk and scalp. Psoriasis is a chronic disease with no cure. Symptoms tend to manifest in cycles, flaring-up for a few weeks or months, and then subsiding or going into remission for a period of time.

Rosacea: a chronic inflammatory skin disease characterized by erythema, papules, telangiectasia, edema, pustules, or a combination of these symptoms. Skin lesions generally occur on the central face, such as the cheeks, forehead, chin, and nose. Individuals with rosacea may also experience rhinophyma, facial flushing, or stinging, pain or burning sensations. Signs and symptoms may flare-up for weeks to months, and then go away for a period of time. The underlying cause of rosacea is unknown, however, abnormalities in the innate immune system and neurovascular dysregulation have been implicated. In addition, certain factors such as demodex (mite) colonization, microbial stimuli, ultraviolet (UV) radiation, heat, and stress may initiate a flare-up or aggravate rosacea. Clinically, rosacea can be categorized into papulopustular, erythematotelangiectatic, ocular, and phymatous rosacea.

Scleroderma: a chronic autoimmune disease affecting the skin and other organs of the body. Scleroderma is characterized by thickening and tightening of the skin along with inflammation and scarring of many body parts, leading to problems in the lungs, kidneys, heart, intestinal system and other areas. Localized scleroderma typically only affects the skin, although it can also affect the muscles, joints and bones. It does not affect internal organs. Symptoms include discolored patches on the skin (morphea); or streaks or bands of thick, hard skin on the arms and legs (linear scleroderma). When linear scleroderma occurs on the face and forehead, it is called en coup de sabre. Systemic scleroderma is the most serious form of the disease, can affect the skin, muscles, joints, blood vessels, lungs, kidneys, heart and other organs. There are two major forms of systemic scleroderma: limited cutaneous systemic sclerosis (formerly called CREST syndrome) and diffuse cutaneous systemic scleroderma.

Skin care composition: a topical composition having active ingredients that can improve the condition, health, aesthetic and/or cosmetic appearance of skin. Such improvements can be, for example, decreasing redness or inflammation of skin. In some implementations, the topical composition disclosed herein is a skin care composition.

Skin flushing or flush: a sudden reddening of the face, neck, or upper chest due to increased blood flow. Skin flushing can be triggered by emotions, such as embarrassment, stress, anger, or excitement. Other triggers include diet (e.g., alcohol use, hot or spicy foods), hormonal changes, hot flashes from menopause, exercise, rapid changes in temperature or heat exposure, and certain medications.

Subject: a living multi-cellular vertebrate organism, a category that includes human and non-human mammals, such as non-human primates. In some examples, the subject is a human. In some implementations, the subject has chronic skin flushing, or an inflammatory skin condition.

Topical composition: a composition formulated for topical application that is generally applied externally to a particular body surface. For example, the topical composition can be topically applied to the skin, such as to a lesion or area of the skin affected by redness or inflammation. In some examples, the topical composition described herein is topically applied to facial skin. A topical composition that is intended for application to the skin is a “skin care composition.”

Transient receptor potential cation channel subfamily M (melastatin) member 8 (TRPM8): a thermally regulated non-selective Ca²⁺-permeable cation channel that is activated by cold temperature or TRPM8 agonists. The channel is a homotetramer, composed of four identical subunits with a transmembrane domain with six helices (S1-6). The first four, S1-4, act as the voltage sensor and allow binding of channel agonists (e.g., menthol or icilin). S5, S6 and a connecting loop make up the pore, a non-selective cation channel which consists of a highly conserved hydrophobic region. Upon activation, the channel allows the entry of Na⁺ and Ca²⁺ ions into the cell, leading to depolarization and the generation of an action potential. The signal is conducted from primary afferents, eventually leading to the sensation of cold. TRPM8 is also known as the cold and menthol receptor 1 (CMR1). It is encoded by the TRPM8 gene (see, e.g., GenBank Gene ID: 79054 (nebi.nlm.nih.gov/genbank/)).

Treating, Treatment, or Therapy: a therapeutic intervention that ameliorates a sign or symptom of a disease or pathological condition, including objective or subjective parameters, such as abatement, remission, slowing the rate of degeneration or decline, or improving a subject's physical or mental well-being. Treatment may be assessed by objective or subjective parameters, including physical examination, patient self-assessment, blood or other clinical tests, and the like. A “prophylactic” treatment is a treatment administered to a subject for the purpose of decreasing the risk of developing a disease or pathological condition.

TRPM8 agonist: an agent that activates TRPM8. Non-limiting examples include: menthol, borneol, linalool, geraniol, hydroxy-citronellal, icilin, WS-12 (preferred IUPAC name: (1R,2S,5R)—N-(4-Methoxyphenyl)-5-methyl-2-(propan-2-yl)cyclohexane-1-carboxamid). Frescolat MGA, Frescolat ML, PMD 38 (also known as p-Menthane-3,8-diol; preferred IUPAC name: 2-(2-Hydroxypropan-2-yl)-5-methylcyclohexan-1-ol), Coolact P, M8-Ag (see, e.g., Patel et al. (2014) Pain, 155(10): 2097-2107), and Cooling Agent 10 (also known as menthoxypropanediol). See also, Behrendt et al., (2004) Br J Pharmacol. 141(4): 737-745.

Ultraviolet light protectant (UV protectant): a compound that can be applied to the skin to block or absorb UV radiation. UV protectants can block or absorb UVA, UVB, or both. Non-limiting examples include aminobenzoic acid, avobenzone, cinoxate, dioxybenzone, drometrizole trisiloxane, homosalate, meradimate, octocrylene, octinoxate, octisalate, oxybenzone, padimate O, para-aminobenzoic acid (PABA), ensulizole, sulisobenzone, titanium dioxide, trolamine salicylate, zinc oxide, ethylhexyl triazone, ecamsule, silatriazole, bemotrizinol, bisoctrizole, iron oxide, red veterinary petrolatum, and kaolin clay.

Unit dose: a physically discrete unit containing a predetermined quantity of an active material calculated to individually or collectively produce a desired effect. A single unit dose or a plurality of unit doses can be used to provide the desired effect or activity, such as decreasing skin redness or inflammation. In another example, the unit dosage form contains multiple predetermined dosages.

Urticaria: a condition commonly known as hives. Urticaria can be triggered by many things, including certain foods, medications, and stress. Symptoms include itchy, raised, red, or skin-colored welts on the skin's surface. Acute urticaria is the most common type. Symptoms typically last less than six weeks and can be caused by insect bites or stings, medications, infections, or consuming certain foods (e.g., nuts, chocolates, fish, tomatoes, eggs, berries or milk). The symptoms of chronic urticaria last more than six weeks. The cause of chronic urticaria is usually difficult to identify, and often remains unknown.

There are a number of physical urticarias, which are urticarias that manifest in response to a physical stimulus. For example, papular urticaria is a condition when individuals develop hives as an allergic reaction to bites from insects like mosquitoes, fleas, mites, carpet beetles or bed bugs. Cholinergic urticaria is a condition when individuals develop hives as an allergic reaction caused by an increase in body temperature. Cold urticaria is a condition when individuals develop hives when exposed to cold, and solar urticaria is a condition when individuals develop hives as due to sun exposure.

### I. Topical Compositions

Disclosed herein are topical compositions that include an effective amount of β-alanine and an effective amount of a TRPM8 agonist. In some implementations, the effective amount of β-alanine is an amount of β-alanine that decreases skin inflammation or redness when applied topically. In some examples, the effective amount of F-alanine decreases dermal mast cell activation when applied topically. In some implementations, the effective amount of the TRPM8 agonist is an amount that decreases transient itch induced by β-alanine. In some implementations, the effective amount of the TRPM8 agonist is an amount that decreases transient itch induced by B1-alanine without causing eye irritation when applied to facial skin (e.g., cheek) of the subject.

The topical composition includes an amount of β-alanine effective to decrease skin inflammation or redness and/or dermal mast cell activation when applied topically to a subject. In some implementations, the topical composition includes about 0.2% to about 20% weight by weight (w/w) β-alanine. In some examples, the topical composition includes about 0.2% to about 19%, about 0.2% to about 18%, about 0.2% to about 17%, about 0.2% to about 16%, about 0.2% to about 15%, about 0.2% to about 14%, about 0.2% to about 13%, about 0.2% to about 12%, about 0.2% to about 11%, about 0.2% to about 10%, about 0.2% to about 9%, about 0.2% to about 8%, about 0.2% to about 7%, about 0.2% to about 6%, about 0.2% to about 5%, about 0.2% to about 4%, about 0.2% to about 3%, about 0.2% to about 2% w/w, about 0.5% to about 20%, about 1% to about 20%, about 2% to about 20%, about 3% to about 20%, about 4% to about 20%, about 5% to about 20%, about 6% to about 20%, about 7% to about 20%, about 8% to about 20%, about 9% to about 20%, about 10% to about 20%, about 11% to about 20%, about 12% to about 20%, about 13% to about 20%, about 14% to about 20%, about 15% to about 20%, about 16% to about 20%, about 17% to about 20%, about 18% to about 20%, or about 19% to about 20%, about 0.5% to about 10%, about 1% to about 10%, about 2% to about 10%, about 3% to about 10%, about 4% to about 10%, about 5% to about 10%, about 6% to 10%, about 7% to 10%, about 8% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 2% to about 8%, about 2% to about 6%, about 4% to about 8%, about 2.5% to about 10%, about 2.5% to about 7.5%, about 2.5% to about 5%, about 5% to about 8%, about 1% to about 18%, about 2% to about 18%, about 5% to about 18%, about 10% to about 18%, about 15% to about 18%, about 5% to about 15%, about 7.5% to about 15%, or about 10% to about 15% w/w β-alanine. In some examples, the topical composition includes 0.2% to 15% w/w β-alanine. In some examples, the topical composition includes 1% to 10% w/w 3-alanine. In some examples, the topical composition includes 5% to 10% w/w β-alanine. In some examples, the topical composition includes 2.5% to 10% w/w 3-alanine. In a further example, the topical composition includes 2.5% to 7.5% w/w β-alanine.

In some implementations, the topical composition includes about 0.2%, about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17% about 18%, about 19%, or about 20% w/w β-alanine. In some examples, the topical composition includes about 5% w/w 1-alanine.

In some examples, the topical composition includes no more than about 1%, about 2%, about 3%, about 4%, about 5%, about 6%, about 7%, about 8%, about 9%, about 10%, about 11%, about 12%, about 13%, about 14%, about 15%, about 16%, about 17% about 18%, about 19%, or about 20% w/w β-alanine. In some examples, the topical composition includes no more than about 15% %-alanine.

The topical composition can include any suitable TRPM8 agonist that can be formulated in a topical composition and is effective at decreasing transient itch induced by the β-alanine in the topical composition when it is applied topically to a subject. Non-limiting examples of suitable TRPM8 agonists include menthol, icilin, crysim-1, borneol, linalool, geraniol, hydroxy-citronellal, WS-12, Frescolat MGA, Frescolat ML, PMD 38, Coolact P, M8-Ag, Cooling Agent 10, or a combination thereof. In some examples, the TRPM8 agonist is menthol, icilin, or a combination thereof. In some implementations, the TRPM8 agonist is menthol.

The topical composition includes an amount of TRPM8 agonist effective to decrease transient itch induced by the β-alanine in the topical composition when applied topically to a subject. In some implementations, the topical composition includes about 0.1% to about 20% weight by weight (w/w) TRPM8 agonist. In some examples, the topical composition includes about 0.1% to about 19%, about 0.1% to about 18%, about 0.1% to about 17%, about 0.1% to about 16%, about 0.1% to about 15%, about 0.1% to about 14%, about 0.1% to about 13%, about 0.1% to about 12%, about 0.1% to about 11%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2% w/w, about 0.1% to about 1% w/w, about 0.1% to about 0.5% w/w, about 0.2% to about 20%, about 0.5% to about 20%, about 1% to about 20%, about 2% to about 20%, about 3% to about 20%, about 4% to about 20%, about 5% to about 20%, about 6% to about 20%, about 7% to about 20%, about 8% to about 20%, about 9% to about 20%, about 10% to about 20%, about 11% to about 20%, about 12% to about 20%, about 13% to about 20%, about 14% to about 20%, about 15% to about 20%, about 16% to about 20%, about 17% to about 20%, about 18% to about 20%, about 19% to about 20%, about 0.2% to about 10%, about 0.5% to about 10%, about 1% to about 10%, about 2% to about 10%, about 3% to about 10%, about 4% to about 10%, about 5% to about 10%, about 6% to about 10%, about 7% to about 10%, about 8% to about 10%, about 9% to about 10%, about 0.2% to about 9%, about 0.2% to about 8%, about 0.2% to about 7%, about 0.2% to about 6%, about 0.2% to about 5%, about 0.2% to about 4%, about 0.2% to about 3%, about 0.2% to about 2%, about 0.2% to about 1%, about 0.2% to about 0.5%, about 0.5% to about 5%, about 0.5% to about 2%, about 0.5% to about 1.5%, about 1% to about 5%, about 2% to about 5%, about 3% to about 5%, about 4% to about 5%, about 2% to about 8%, about 2% to about 6%, about 4% to about 8%, about 5% to about 8%, about 1% to about 18%, about 2% to about 18%, about 5% to about 18%, about 10% to about 18%, about 15% to about 18%, about 5% to about 15%, about 7.5% to about 15%, or about 10% to about 15% w/w TRPM8 agonist. In some examples, the TRPM8 agonist is menthol. In some examples, the topical composition includes 0.1% to 5% w/w menthol. In some examples, the topical composition includes 0.1% to 2% w/w menthol. In some examples, the topical composition includes 0.2% to 1% w/w menthol. In further examples, the topical composition includes 0.5% to 1.5% w/w menthol.

In some implementations, the topical composition includes about 0.1%, about 0.2%, about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17% about 18%, about 19%, or about 20% w/w TRPM8 agonist. In some examples, the TRPM8 agonist is menthol. In some examples, the topical composition includes about 0.5% w/w menthol.

In other implementations, the topical composition includes no more than about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17% about 18%, about 19%, or about 20% w/w TRPM8 agonist. In some examples, the TRPM8 agonist is menthol. In some examples, the topical composition includes no more than about 5% menthol. In another example, the topical composition includes no more than about 1% menthol.

In non-limiting examples, the topical composition includes about 0.2% to about 15% w/w β-alanine and about 0.1% to about 5% w/w menthol. In some examples, the topical composition can include about 0.2% to about 15% w/w β-alanine and about 0.1% to about 2% w/w menthol. In some examples, the topical composition includes about 0.2% to about 15% w/w β-alanine and about 0.2% to about 1% w/w menthol. In some examples, the topical composition includes about 1% to about 10% w/w f-alanine and about 0.1% to about 5% w/w menthol. In some examples, the topical composition includes about 1% to about 10% w/w β-alanine and about 0.2% to about 2% w/w menthol. In some examples, the topical composition includes about 5% to about 10% w/w β-alanine and about 0.1% to about 5% w/w menthol. In some examples, the topical composition includes about 5% to about 10% w/w β-alanine and about 0.1% to about 2% w/w menthol. In some examples, the topical composition includes about 5% to about 10% w/w J-alanine and about 0.2% to about 1% w/w menthol. In some examples, the topical composition includes about 2.5% to about 7.5% w/w β-alanine and about 0.5% to about 1.5% w/w menthol.

In some implementations, the topical composition includes an amount of f-alanine and menthol that is useful for decreasing redness or inflammation when administered to skin affected by redness or inflammation.

In some implementations, the disclosed topical composition further includes additional ingredients, such as a topical UV protectant, antibiotic, antiparasitic, steroid (e.g., corticosteroid), non-steroidal anti-inflammatory agent, calcineurin inhibitor, JAK inhibitor, analgesic, α2-adrenergic receptor agonist, α1A-adrenoceptor agonist, vitamins (e.g., vitamin A, C, or E), ceramides, extracts or other natural products, or combinations thereof.

Non-limiting examples of suitable topical UV protectants include aminobenzoic acid, avobenzone, cinoxate, dioxybenzone, drometrizole trisiloxane, homosalate, meradimate, octocrylene, octinoxate, octisalate, oxybenzone, padimate O, para-aminobenzoic acid (PABA), ensulizole, sulisobenzone, titanium dioxide, trolamine salicylate, zinc oxide, ethylhexyl triazone, ecamsule, silatriazole, bemotrizinol, bisoctrizole, iron oxide, red veterinary petrolatum, and kaolin clay. The UV protectant can be added in an amount that provides a sun protection factor (SPF) of at least 5, for example, about SPF 5, about SPF 10, about SPF 15, about SPF 20, about SPF 25, about SPF 30, about SPF 35, about SPF 40, about SPF 45, about SPF 50, about SPF 55, about SPF 60, about SPF 65, about SPF 70, about SPF 75, about SPF 80, or more. In some examples, the UV protectant is added in an amount that provides a sun protection factor (SPF) of 5 to 80. In some examples, the UV protectant is added in an amount that provides a sun protection factor (SPF) of 15 to 80. In some examples, the UV protectant is added in an amount that provides a sun protection factor (SPF) of 15 to 40.

Topical antibiotics are antibiotics applied to a body surface. Antibiotics can target bacteria, fungus or any other microorganism. Antibiotics can contain active agents such as macrolide antibiotic (such as erythromycin), a sulfa antibiotic (such as sulfacetamide), a cyclic peptide (such as bacitracin a polymyxin) a psuedomonic acid (such as mupirocin), an ammyroglycoside (such as neomycin), or a quinolone (such as ciprofloxacin or ofloxacin), a nitroimidazole (such as metronidazole), or a combination of active ingredients (such as bacitracine/polymyxin or neomycin/polymyxin B/bacitracin). The antibiotic can be narrow or broad spectrum. “Narrow-spectrum” antibacterial antibiotics target specific types of bacteria, such as Gram-negative or Gram-positive bacteria. “Broad-spectrum antibiotics” affect a number of different types of bacteria. Antibacterial agents also include cyclic lipopeptides (such as daptomycin), glycylcyclines (such as tigecycline), and oxazolidinones (such as linezolid). In some examples, the topical antibiotic is an azole antifungal, for example, ketoconazole, itraconazole, clotrimazole, voriconazole, posaconazole, or miconazole. In some examples, the topical antibiotic is clindamycin, metronidazole, cyclosporine, or azathioprine.

Antiparasitics are used in the management and treatment of infections by parasites, such as protozoa, helminths, and ectoparasites (e.g., fleas, lice, or mites). Suitable topical antiparasitics include, but are not limited to, ivermectin, lindane, permethrin, benzyl alcohol, malathion, and crotamiton.

Suitable topical steroids include amcinonide, beclomethasone, betamethasone, clobetasol, clobetasone, desonide, desoximetasone, diflorasone, fluocinolone, fluocinonide, flurandrenolide, fluticasone, halcinonide, halobetasol, hydrocortisone, mometasone, prednicarbate, and triamcinolone. In some implementations, the steroid is a corticosteroid. Exemplary corticosteroids include hydrocortisone, clobetasone, beclomethasone, betamethasone, fluticasone, clobetasol, and mometasone.

Non-limiting examples of suitable non-steroidal anti-inflammatory agents include azelaic acid, non-steroidal anti-inflammatory drugs (NSAIDs), and PDE4 inhibitors. NSAIDs have anti-inflammatory, analgesic, and antipyretic effects. Although different NSAIDs have different structures, they all target (block) cyclooxygenase (COX) enzymes. Exemplary NSAIDs that can be applied topically include diclofenac (including diclofenac sodium and diclofenac hydroxyethylpyrrolidine), ibuprofen, ketoprofen, felbinac, and piroxicam. PDE4 inhibitors modulate the immune system by inhibiting phosphodiesterases-4 (PDE4), which helps prevent and reduce inflammation. An exemplary topical phosphodiesterase-4 (PDE4) inhibitor is crisaborole.

The topical compositions can include one or more topical calcineurin inhibitor. Calcineurin inhibitors are immunosuppressive drugs that block T-cell proliferation by inhibiting the signaling phosphatase calcineurin. Exemplary topical calcineurin inhibitors include cyclosporine, tacrolimus, and pimecrolimus.

The topical compositions can include one or more topical JAK inhibitor (or JAK-STAT pathway inhibitor). Non-limiting examples of JAK inhibitors include abrocitinib, baricitinib, ruxolitinib, and upadacitinib. The topical composition disclosed herein can also include a topical α2-adrenergic receptor agonist (e.g., brimonidine), α1A-adrenoceptor agonist (e.g., oxymetazoline), or combinations thereof.

The topical composition can include vitamins. Non-limiting examples of vitamins include Vitamin A (retinol and its analogues, such as retinyl palmitate), any of the 12 B-complex vitamins (e.g., thiamine (B1), riboflavin (B2), niacin (B3), pantothenic acid (B5), B6, biotin (B7), folate (B9), B12, and their analogs), Vitamin C (and its analogues, such as magnesium ascorbyl phosphate, ascorbyl palmitate, etc.), Vitamin D (e.g., calcitriol (D3)), and Vitamin E (tocopherol and its analogues, such as tocopheryl acetate).

The topical composition can also include natural or synthetic ceramides, essential fatty acids (e.g., omega-3 or omega-6 (e.g., linoleic acid)), glycerin, glycols, polyols, hyaluronic acid, sodium PCA, or other moisturizing ingredients.

In some implementations, the topical composition includes a natural product, such as a botanical extract (including tinctures), for example, from Arnica montanais (commonly known as wolf s bane, leopard's bane, mountain tobacco, and mountain arnica), Symphytum (comfrey), Sesamum indicum (sesame), soy, tea (green, black, or white), Matricaria recutita (chamomile), Coffea arabica (coffee or coffeeberry), almond, angelica root, acacia, aloe, althea anise, apple, avocado, basil, bayberry, bilberry, blue malva flower, blackberry, burdock, borage, bergamot, buchu, cannabis, cranberry, clover blossom, calendula, clove, carrot, China bark, cinnamon, clary sage, coltsfoot, cornflower, cranesbill, cucumber, dandelion, dulse, eucalyptus, echinacea, elderberry, fennel, fenugreek, garlic, gentian root, geranium, ginko biloba, ginseng, grapefruit, grapeseed, goldenseal, hops, hawthorn, henna, horse chestnut, horsetail, hibiscus, ivy, Irish moss, jasmine, juniper berry, kelp, kiwi, linden flower, lovage, lavender, lemongrass, lemon peel, myrrh, mallow, meadow sweet (mayflower), melon, mint, melilot (hayflower), milk thistle, nettle, oatmeal, orange flower, orange peel, oak moss, peach, plantain, parsley, peppermint, primrose, papaya, quassia, quince, rose bud, rose hips, rosemary, sage, sambucus elder, slippery elm bark, spearmint, sambucus, sandalwood, southernwood, strawberry, sunflower, St. Johns wort, tangerine, tansy, thyme, tomato, uva ursi (bearberry), violet, white lily, valerian root, watercress, walnut (black), white birch, white willow bark, wild indigo, witch hazel, yucca, yarrow, or other botanical source.

The topical compositions can include synthetic or naturally derived phytochemicals, such as sesquiterpene lactones, rosmarinic acid, sesamin, sesamol, sesaminol, sesamolin, isoflavones (e.g., genistein and daidzein), polyphenolics (e.g., chlorogenic acid, ferrulic acid, quinic acid, and condensed proanthocyanidins), epigallocatechin-3-gallate (EGCG), flavonoids (e.g., quercetin, theaflavin, kaempferol, apigenin, luteolin, rutin), catechins, terpenoids (e.g., bisoprolol, matricine, levomenol, chamazulene), caffeine, or combinations thereof.

The topical compositions can include one or more topical analgesics and/or anesthetics. An analgesic is a medication that relieves pain. An anesthetic is a substance that induces a temporary loss of sensation, which can include loss of sensation of pain or itching. Non-limiting examples of suitable topical analgesics include capsaicin, diclofenac, lidocaine, methyl salicylate, and trolamine salicylate. Exemplary topical anesthetics include lidocaine, pramoxine, benzocaine, dibucaine, and camphor. In some examples, the topical composition does not include camphor, lidocaine, methyl salicylate, pramoxine, benzocaine, dibucaine, or diclofenac. In some examples, the topical composition does not include a topical anesthetic or analgesic other than a TRPM8 agonist (excludes topical anesthetics or analgesics that are not TRPM8 agonists). In some examples, the topical composition does not include a topical anesthetic or analgesic other than menthol.

In some implementations, the topical composition does not include a topical steroid (e.g., amcinonide, beclomethasone, betamethasone, clobetasol, clobetasone, desonide, desoximetasone, diflorasone, fluocinolone, fluocinonide, flurandrenolide, fluticasone, halcinonide, halobetasol, hydrocortisone, mometasone, prednicarbate, and triamcinolone).

The topical composition can be formulated in any form suitable for application to a surface of the body (i.e., skin), such as a cream, lotion, gel, ointment, paste, soap, shampoo, solution, suspension, spray, bioadhesive, paints, or the like. Methods of producing cosmetically acceptable topical compositions, such as creams, ointments, lotions, sprays, gels, paste, aqueous solutions, or suspensions are described, for example in Remington: The Science and Practice of Pharmacy, 23^{rd} Ed. (Academic Press), 2021 (ISBN 9780128200070).

The topical composition can be formulated as a cream. Creams are viscous liquids or semisolid emulsions, either oil-in-water or water-in-oil. Cream bases are often water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

The topical composition can be formulated as a lotion. Lotions are preparations to be applied to the skin surface, generally without friction, and are typically liquid or semiliquid preparations in which particles, including the active agent, are present in a water or alcohol base. Lotions are usually suspensions of solids, and preferably, comprise a liquid oily emulsion of the oil-in-water type. Lotions can be used for large body areas, because of the ease of applying a more fluid composition. It is generally necessary that the insoluble matter in a lotion be finely divided. Lotions typically contain suspending agents to produce better dispersions as well as compounds useful for localizing and holding the active agent in contact with the skin, e.g., methylcellulose, sodium carboxymethyl-cellulose, or the like.

The topical composition can be formulated as a gel. Gels are semisolid, suspension-type systems. Single-phase gels contain organic macromolecules distributed substantially uniformly throughout the carrier liquid, which is typically aqueous, but also may contain an alcohol or an oil. Useful “organic macromolecules,” specifically gelling agents, are crosslinked acrylic acid polymers such as the “carbomer” family of polymers, e.g., carboxypolyalkylenes that are commercially available as CARBOPOL®. Also of use are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

The topical composition can be formulated as an ointment. Ointments are semisolid preparations that are typically based on petrolatum or petroleum derivatives. The specific ointment base to be used can be selected by a practitioner based on desired characteristics, e.g., emolliency or the like. An ointment base is generally inert, stable, non-irritating, and non-sensitizing. Ointment bases are grouped in four classes: oleaginous bases; emulsifiable bases; emulsion bases; and water-soluble bases (see, e.g., Remington: The Science and Practice of Pharmacy, 23rd Ed. (Academic Press), 2021 (ISBN 9780128200070)). Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, acetyl alcohol, glyceryl monostearate, lanolin, and stearic acid. Water-soluble ointment bases are prepared from polyethylene glycols of varying molecular weight.

The topical composition can be formulated as a paste. Pastes are generally semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

The topical composition can be formulated as soap. Soap is a solid, liquid, or cream product used in conjunction with water for washing and cleaning. It is usually produced in a solid molded form (bar soap) but may also come in the form of liquids or creams dispersed from dispensers. Soaps typically contain surfactants that, when applied to a soiled surface in combination with water wet the dirt and effectively holds particles in suspension so it can be rinsed off with clean water. Animal based oils/fats, such as lard and tallow, were traditionally used to make soaps. However, commercial soaps typically use a vegetable base oil. Common vegetable base oils include olive, coconut, palm kernel, palm, castor, apricot, avocado, almond, jojoba, hemp, or other nut or seed oils, or butters such as cocoa, mango or shea butter. Lye is reacted with the base oil to create soap (saponification). Examples of lye include sodium hydroxide and potassium hydroxide. Sodium hydroxide is used to make solid soap, while potassium hydroxide is used to make liquid soaps. A combination of the two is used to make cream soaps. Soaps can be used as a deodorant bar, or to cleanse and moisturize.

The topical composition can be formulated as a shampoo (including other hair rinses). A shampoo is typically a viscous liquid preparation containing detergent or soap for washing the hair. Less commonly, shampoo is available in bar form, like a bar of soap. Shampoos include surfactants, such as ammonium lauryl sulfate, sodium lauryl sulfate, sodium laureth sulfate, decyl glucoside and lauryl glucoside. Shampoos can also include foam boosters (e.g., lauramide DEA or cocamidopropyl betaine), thickeners (e.g., methylcellulose or carbomer), conditioning agents (e.g., polyquaternium-10, guar hydroxypropyltrimonium chloride, dimethicone, cyclomethicone, or quaternium 80), preservatives (e.g., DMDM hydantoin, methylparaben, sodium benzoate, benzyl alcohol and phenoxyethanol), and well as other ingredients such as dyes, fragrances, pH buffers, chelating agents, opacifying agents, vitamins, proteins, and herbal extracts. Shampoos can also include active ingredients, such as zinc pyrithione, salicylic acid, ketoconazole, or selenium sulfide.

The disclosed topical compositions include solutions. Solutions are generally homogeneous mixtures prepared by dissolving one or more chemical substances (solutes) in a liquid such that the molecules of the dissolved substance are dispersed among those of the solvent. The solution may contain other cosmetically acceptable chemicals to buffer, stabilize and/or preserve the solute. Common examples of solvents used in preparing topical solutions are ethanol, water, propylene glycol or any other acceptable vehicles. These can be applied in any manner, such as spraying them on the skin, painting them on the skin, or wetting a bandage with the solution.

A bioadhesive is a composition that adheres, or causes adhesion, in living tissue. In clinical applications, bioadhesives can be used as tissue adhesives, hemostats, tissue sealants, wound dressings, delivery vehicles, for medical device fixation, or other applications. Compared with traditional wound closure methods, including sutures, wires, or staples, bioadhesives have less potential for damaging tissues and can promote wound healing through different mechanisms. For example, bioadhesives can possess antibacterial, anti-inflammatory, and antioxidant properties. Cyanoacrylate-based bioadhesives are the most widely used tissue adhesives for wound closure. Examples of bioadhesives include FocalSeal®, DuraSeal®, Coseal®, Tisseel®, and Bioglue®.

The disclosed topical compositions may include a cosmetically acceptable viscosity enhancer and/or film former. A viscosity enhancer increases the viscosity of the formulation so as to inhibit its spread beyond the site of application. Balsam Fir (Oregon) is an example of a suitable viscosity enhancer. A film former, when it dries, forms a protective film over the site of application. The film inhibits removal of active ingredients and keeps it in contact with the site. An example of a film former that is suitable for use in this invention is Flexible Collodion, USP (see Remington: The Science and Practice of Pharmacy, 23rd Ed. (Academic Press), 2021 (ISBN 9780128200070)). Collodions are ethyl ether/ethanol solutions containing pyroxylin (a nitrocellulose) that evaporate to leave a film of pyroxylin. A film former may act additionally as a carrier. Solutions that dry to form a film are sometimes referred to as paints.

A cream, lotion, gel, ointment, paste, soap, shampoo, bioadhesive, paint, and the like may be administered to an affected surface of a subject, for example, spread on skin of a subject affected by redness or inflammation (although shampoo is typically applied specifically to the hair and scalp). A solution may be administered similarly, but more typically will be applied with a dropper, swab, sprayer or the like, to the affected areas. Application methods generally depend on viscosity of the composition to be applied. The composition can be applied directly to the target location, for example in a topical preparation such as an ointment, or as a part of a dressing or a bandage. The composition can be formulated as a unit dosage, for administration by a device to the skin. The unit dosage may be a reservoir of the topical composition in a carrier, for example an adhesive carrier capable of adhering to the skin for a desired period of time, such as at least a day or more.

The topical composition can be formulated as an emollient (moisturizer). Emollients soften and moisturize skin and can be used to decrease itching or flaking. Dry skin can be caused by water loss in the upper layer of the skin. Emollients work by forming an oily layer on the top of the skin to trap water in the skin. Petrolatum, lanolin, mineral oil and dimethicone are common emollients. Humectants, including glycerin, lecithin, and propylene glycol, draw water into the outer layer of skin. Many emollient products also have ingredients that soften keratin, such as urea, alpha hydroxy acids such as lactic/citric/glycolic acid, and allantoin. Compounds, such as zinc oxide or white petrolatum, that protect the skin against irritation can also be included.

The topical composition can be formulated for application to any external part of the body. In some non-limiting examples, the topical composition is formulated for application to the head, face (e.g., cheek, nose, forehead, etc.), scalp, arms, hands, legs, feet, or body. In some implementations, the topical composition is formulated for application to the face (facial application).

The topical composition can be used in combination with an occlusive overlayer so that moisture evaporating from the body surface is maintained within the formulation upon application to the body surface and thereafter. In some examples, the topical composition is used in combination with a wet dressing (e.g., wet wrap therapy). For wet dressings, gauze, bandages, or the like, are soaked in water and applied to an affected area of skin, for example, after administering the topical composition. A dry layer is then often wrapped over the wet layer. The bandages are left on for several hours, such as overnight.

The topical composition can be formulated as a concentrate that can be used as an additive in existing skin care products. The concentrate may be formulated such that when a specified amount is added to a particular amount of skin care product, the resulting concentration of β-alanine and TRPM8 agonist is a desired concentration (an effective amount).

### II. Methods of Use

Also disclosed are methods for decreasing skin inflammation or redness in a subject and methods of reducing frequency, duration, or severity of signs or symptoms of an inflammatory skin condition. The method includes administering an effective amount of the topical composition disclosed herein to an affected area of skin of the subject. In some implementations, the topical composition is used in a method of decreasing skin inflammation or redness in a subject. The method includes administering an effective amount of the topical composition disclosed herein to an affected area of skin on the subject. The affected area may be an area that has signs or symptoms of inflammation or redness, or it may be an area that is at risk of developing signs or symptoms of inflammation or redness, for example, an area of skin that is prone to inflammation or redness or has historically had inflammation or redness. In some implementations, the subject has an inflammatory skin condition or chronic skin flushing.

In some implementations, the affected area of skin is affected by an inflammatory skin condition, such as rosacea, acute or chronic urticaria, dermatographia, mastocytosis, dermatitis, acne, psoriasis, or scleroderma. In some implementations, the affected area of skin is affected by chronic skin flush, for example, chronic skin flushing induced by exercise, diet (e.g., alcohol, nuts, shellfish, etc.), hormone changes, menopause, or emotion (e.g., stress, embarrassment or excitement). In some examples, the chronic skin flushing is alcohol flush reaction. In some implementations, the affected area of skin is affected by a wound (e.g., skin abrasion). In some implementations, the method includes selecting a subject with an inflammatory skin condition or chronic skin flushing for administration of the topical composition.

In some implementations, a subject having an inflammatory skin condition, such as rosacea, acute or chronic urticaria, dermatographia, mastocytosis, dermatitis, acne, psoriasis, or scleroderma, is selected for application of the disclosed topical composition.

Topical compositions that decrease redness can be identified by a reduction in redness relative to a suitable control (e.g., a pre-administration measurement from the subject or administration of a blank (empty) vehicle). A reduction in redness of the skin can be measured subjectively (qualitative) or objectively (quantitative). Topical compositions that decrease inflammation may be identified by a decrease in dermal mast cell activity in the subject following administration of the topical composition to the skin, and consequently a decrease in inflammatory mediators, such as cytokines, histamine, or serotonin, relative to a suitable control (e.g., a pre-administration measurement from the subject, administration of a blank (empty) vehicle, or a historic reference of “normal” values). Decreased inflammation may also be identified visually as a decrease in redness of areas of skin administered the topical composition. In some implementations, an effective amount of the topical composition decreases the duration, frequency, or severity of skin inflammation or redness.

In some implementations, administration of the topical composition decreases redness or inflammation by at least 5%, for example, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or even 100% (redness or inflammation substantially resolves). In some examples, redness or inflammation is decreased by about 5% to 100%, for example, by about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 10% to about 60%, about 10% to about 70%, about 10% to about 80%, about 20% to about 30%, about 20% to about 40%, about 20% to about 50%, about 20% to about 60%, about 20% to about 70%, about 20% to about 80%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 80%, about 40% to about 100%, about 50% to about 75%, about 50% to about 100%, about 60% to about 100%, about 70% to about 100%, about 75% to about 100%, about 85% to about 100%, about 90% to about 100%, or about 95% to about 100% (redness or inflammation substantially resolves).

In some implementations, the method decreases frequency, duration, or severity of signs or symptoms of an inflammatory skin condition (e.g., rosacea, acute or chronic urticaria, dermatographia, mastocytosis, dermatitis, acne, psoriasis, or scleroderma) in the subject. In some implementations, the method includes selecting a subject with an inflammatory skin condition and administering an effective amount of the topical composition disclosed herein to an area of skin of the subject affected by the inflammatory skin condition. In some examples, the inflammatory skin condition is rosacea. In a non-limiting example, the method includes selecting a subject that has rosacea, and administering an effective amount of the topical composition to an area of skin affected by the rosacea. The subject can have any type of rosacea. In some examples, the subject has papulopustular rosacea, erythematotelangiectatic rosacea, or phymatous rosacea.

The affected area may be an area that has signs or symptoms of the inflammatory skin condition, or it may be an area that is at risk of developing signs or symptoms of the inflammatory skin condition, for example, an area of skin that is prone to symptoms, or has historically had a flare-up. In some examples, the affected area is an area that has signs or symptoms of rosacea, or is an area at risk of developing signs or symptoms of rosacea, for example, an area of skin that is prone to rosacea, or has historically had a rosacea flare-up.

A reduction in the frequency, duration, or severity of signs or symptoms of the inflammatory skin condition is measured relative to a suitable control (e.g., a pre-administration measurement from the subject, application of a blank (empty) vehicle, or a historic reference of “normal” values). A reduction in signs or symptoms can include, for example, reducing lesion size, lesion number, itching (pruritus), redness, inflammation, skin thickness, raw or cracked skin, sores, or duration of a flare up. In some examples, administration of the topical composition decreases signs or symptoms of the inflammatory skin condition by at least 5%, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or even 100% (signs or symptoms substantially resolve). In some examples, signs or symptoms are decreased by about 5% to 100%, for example, by about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 10% to about 60%, about 10% to about 70%, about 10% to about 80%, about 20% to about 30%, about 20% to about 40%, about 20% to about 50%, about 20% to about 60%, about 20% to about 70%, about 20% to about 80%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 80%, about 40% to about 100%, about 50% to about 75%, about 50% to about 100%, about 60% to about 100%, about 70% to about 100%, about 75% to about 100%, about 85% to about 100%, about 90% to about 100%, or about 95% to about 100% (signs or symptoms substantially resolve).

In some implementations, a reduction in the frequency of signs or symptoms includes increasing the duration of symptom remission (time between symptomatic flare-ups). In some examples, a new flare-up will not occur for about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 4 months, about 5 months, about 6 months, about 7 months, about 8 months, about 9 months, about 10 months, about 11 months, or about 12 months, or longer following administration of the topical composition. In some examples, a new flare-up will not occur for about 1 to about 2 weeks, about 1 to about 3 weeks, about 1 to about 4 weeks, about 1 to about 6 weeks, about 1 to about 12 weeks, about 2 to about 4 weeks, about 2 to about 6 weeks, about 2 to about 8 weeks, about 2 to about 12 weeks, about 4 to about 6 weeks, about 4 to about 8 weeks, about 4 to about 12 weeks, about 6 to about 12 weeks, about 1 to about 4 months, about 1 to about 6 months, about 1 to about 12 months, about 2 months to about 4 months, about 2 months to about 6 months, about 2 months to about 8 months, about 2 months to about 12 months, about 4 months to about 6 months, about 4 months to about 8 months, about 4 months to about 12 months, or about 6 months to about 12 months.

The subject can be any mammalian subject, including human or non-human mammals. In some implementations, the subject is a human. The subject can be a child or an adult, for example, a child under the age of 18 or an adult age 18 or older. In some examples, the child is under 5, under 12, or under 16 years of age. In further examples, the child is about 1 to about 3, about 1 to about 5, about 1 to about 12, about 3 to about 5, about 5 to about 10, about 5 to about 12, about 5 to about 16, about 5 to about 18, about 10 to about 12, about 10 to about 16, about 10 to about 18, about 12 to about 16, about 12 to about 18. In some examples, the adult is over about 18, over about 25, over about 30, over about 35, over about 40, over about 45, over about 50, over about 55, over about 60, over about 65, over about 70, or over about 75 years of age. In further examples, the adult is about 18 to about 25, about 18 to about 35, about 18 to about 65, about 20 to about 35, about 25 to about 65, about 35 to about 65, about 40 to about 65, about 40 to about 80, about 65 to about 80, or over about 80. In some examples, the subject is a female adult over the age of 30. In some examples, the subject is a female adult with menopause.

Appropriate dosage can depend on the subject (e.g., human or non-human mammal), age, general condition of the subject, and/or the severity of symptoms, among other factors. Thus, an “effective amount” of the topical composition will fall in a relatively broad range that can be determined through testing and trials. The method can include measuring an outcome, for example, redness, inflammation, skin thickness, skin texture, skin dryness, lesion size, number of lesions, or itching (pruritus).

The topical composition may be administered as a single administration or multiple administrations. In some implementations, the topical composition is administered as needed. For example, twice weekly, three times weekly, four times weekly, five times weekly, once daily, twice daily, three times daily, four times daily, or more. Administration may be over a course of hours, days, months, or years. In some implementations, the topical composition is administered for at least 7 days, for example, at least 10 days, at least 14 days, at least 20 days, at least 24 days, at least 28 days, at least 32 days, at least 36 days, or longer. In some examples, the topical composition is administered for at least 7 days. In some implementations, the topical composition is administered for at least one week, for example, at least two weeks, at least 3 weeks, at least 4 weeks, at least 5 weeks, at least 6 weeks, at least 7 weeks, at least 8 weeks, at least 12 weeks, or longer. In some examples, the topical composition is administered during the remainder of the subject's lifetime. In some examples, the topical composition is administered once per day for one week, two weeks, three weeks, four week, six weeks, eight weeks, twelve weeks, or longer. In some examples, the topical composition is administered at least four times a week for one week, two weeks, three weeks, four week, six weeks, eight weeks, twelve weeks, or longer. In some examples, the topical composition is administered at least three times a week for one week, two weeks, three weeks, four week, six weeks, eight weeks, twelve weeks, or longer. In a non-limiting example, the topical composition is administered once a day for the remainder of the subject's lifetime.

In some implementations, the method further includes administering a topical UV protectant, antibiotic, steroid (e.g., corticosteroid), non-steroidal anti-inflammatory agent, calcineurin inhibitor, JAK inhibitor, analgesic, α2-adrenergic receptor agonist, α1A-adrenoceptor agonist, vitamins (e.g., vitamin A, B, C, D, or E), or combinations thereof, to the subject. Exemplary topical UV protectant, antibiotic, steroid (e.g., corticosteroid), non-steroidal anti-inflammatory agent, calcineurin inhibitor, JAK inhibitor, analgesic, α2-adrenergic receptor agonist, α1A-adrenoceptor agonist, vitamins are provided herein. The additional active ingredient can be administered prior to, during, and/or following administration of the topical composition.

### EXAMPLES

The following examples are provided to illustrate particular features and/or implementations. These examples should not be construed to limit the disclosure to the particular features or implementations described.

### Example 1

### Optimization of β-Alanine Concentration

β-alanine powder (GNC, #369898) was mixed into Versapro® Cream at a concentration of 0%, 2.5%, 5.0% or 7.5% w/w. The ingredients of Versapro® Cream include water, aloe Barbadensis leaf juice, cyclopentasiloxane, disodium EDTA, emulsifying wax NF, ethylhexglyglycering, ethylhexyl stearate, phenoxyethanol, sorbitol, and tocopheryl acetate. The mixture was agitated until homogenous.

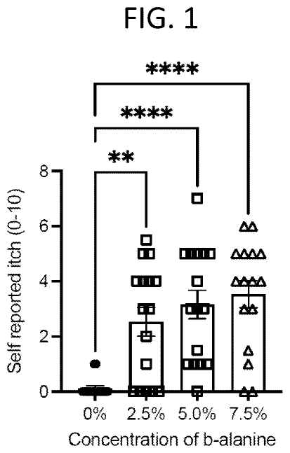

A cohort of patients without skin disease were selected for testing (n=16). Each patient had 0.1 ml of cream applied to cheek skin by a physician. Over the course of 5 minutes, the patients reported the intensity of pruritus (itching) on a scale of 0 (no itching) to 10 (maximal itching). The process was repeated for all four formulations (0%, 2.5%, 5.0% or 7.5% w/w β-alanine) using different areas of cheek skin. The efficacy of topical J3-alanine was determined based on the development of pruritus. While pruritus was evident at the 2.5%, 5.0%, and 7.5% doses, itch was maximal at the 5.0% with no further increase noted with 7.5% (FIG. 1). Thus, 5% β-alanine concentration was selected for further study. Pruritus triggered by topical f3-alanine cream was transient, lasting less than 5 minutes.

### Example 2

### Optimization of Menthol Concentration

While β-alanine has beneficial effects when applied to skin, topical application also induces transient pruritus, limiting its potential use and application. Menthol was selected for testing as an agent that could mask short-term pruritus when added to a topical composition with β-alanine. Since menthol can cause eye irritation, the following study was designed to determine whether a tolerable concentration of menthol is capable of providing relief from β-alanine induced pruritis.

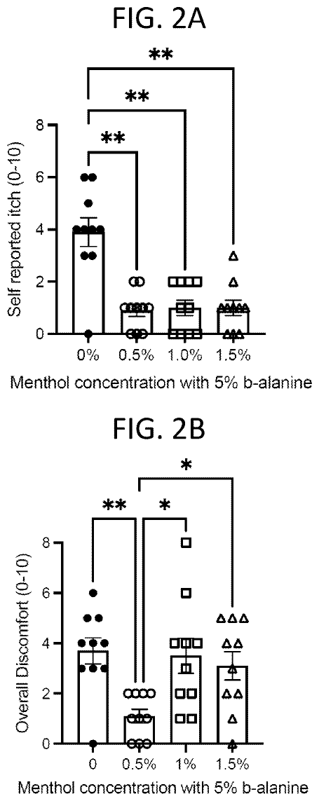

A cohort of patients without inflammatory skin conditions (n=10) was given four different topical formulations for facial application. All four formulations contained 5% w/w β-alanine in Versapro® Cream (see, e.g., Example 1), however, each contained a different concentration of menthol (0.0%, 0.5%, 1.0%, and 1.5% w/w). A physician sequentially applied 0.1 ml of each formulation to distinct areas of the cheek with a 10 minute pause between each application. Patients were asked to rate the degree of itching and overall discomfort on a scale of 0 (no itching) to 10 (maximal itching) over the course of 5 minutes. The same process was repeated for the 0.5%, 1.0% and 1.5% w/w menthol containing formulations. Itching was notably reduced at all concentrations of menthol (FIG. 2A). However, overall discomfort increased with β-alanine cream containing 1.0% and 1.5% menthol, which was attributable to eye irritation and an unpleasant cold sensation (FIG. 2B).

The menthol concentration of 0.5% successfully decreased the intensity of itching without promoting eye discomfort, and will be selected for further testing.

### Example 3

### Clinical Trial Example—Split Face

The following describes an exemplary clinical trial design to test topical application of various concentrations of β-alanine with 0.5% w/w menthol in patients with rosacea. While testing in patients with rosacea is provided as an example, patients with other conditions, such as chronic flushing or an inflammatory skin condition, could be selected and tested in a similar manner.

A cohort of patients with a history of chronic rosacea facial flushing (>6 mo. duration, frequency >2×/week based on historical reporting) are selected. On intake, physicians evaluate each patient's baseline condition using photos, taking measurements of redness, and performing a clinician's Erythema Assessment. Physicians interview patients to identify triggers, family history, and character of flushing (e.g., burning sensation).

Test compositions are produced by mixing 1%, 2%, or 5% w/w β-alanine with 0.5% w/w menthol in any suitable lotion or cream as a base, such as OLAY® complete lotion moisturizer (see, e.g., Example 1). The vehicle is the lotion or cream base without β-alanine and menthol. Patients are instructed to apply the vehicle to % the face (e.g., left side of face) and apply a test composition (either 1%, 2%, or 5% β-alanine with 0.5% menthol) to the other M of the face (e.g., right side of face) once daily for 4 weeks. Patients and physicians are double blinded throughout the study.

Patients self-photograph the face weekly at a set time (e.g., Sunday night after make-up removal). Patients also self-photograph whenever a flare-up is noticed. Patients keep a daily diary of events after application, and note the frequency of flushing, as well as any sensations (e.g., burning) experienced during flushing episodes with an emphasis on right vs. left side of the face (a patient self-assessment (PSA)).

After 4 weeks, a physician evaluates the patient's condition using photography, taking measurements of redness, and performing a clinician's Erythema Assessment. The focus is on comparing the right vs. left side of face.

A similar design could be used to test different concentrations of menthol, for example, instead of testing 0.5% w/w menthol with varying concentrations of β-alanine as described above, a set concentration of β-alanine (e.g., 5% w/w) with varying concentrations of menthol (e.g., 0, 0.25%, 0.5%, 1%, 1.5%, or 2% w/w) can be tested in a similar manner. In addition, other TRPM8 agonists can be substituted for menthol and tested similarly as described here.

### Example 4

### Clinical Trial Example—Whole Face

The following describes an exemplary clinical trial design to test topical application of various compositions in patients with rosacea. While testing in patients with rosacea is provided as an example, patients with other inflammatory skin conditions could be selected and tested in a similar manner.

A cohort of patients with a history of chronic rosacea facial flushing (>6 mo. duration, frequency >2×/week based on historical reporting) are selected for study. On intake, physicians evaluate each patient's baseline condition using photos, taking measurements of redness, and performing a clinician's Erythema Assessment. Physicians interview patients to identify triggers, family history, and character of flushing (e.g., burning sensation).

Test compositions are produced by mixing 1%, 2%, or 5% w/w β-alanine with 0.5% w/w menthol in any suitable lotion or cream as a base, such as OLAY® complete lotion moisturizer (see, e.g., Example 1). The vehicle is the lotion or cream base without β-alanine and menthol. Patients are instructed to apply either vehicle or a test composition (1%, 2%, or 5% w/w β-alanine with 0.5% w/w menthol) to the entire face once daily for 4 weeks. Patients and physicians will be double blinded throughout the study.

Patients self-photograph the face weekly at a set time (e.g., Sunday night after make-up removal). Patients also self-photograph whenever a flare-up is noticed. Patients keep a daily diary of events after application, and note the frequency of flushing as well as any other sensations (e.g., burning) experienced during flushing episodes (a patient self-assessment (PSA)).

After 4 weeks, a physician evaluates each patient's condition using photography, taking measurements of redness, and performing a clinician's Erythema Assessment. The main outcome will be evaluating the number and intensity of flushing episodes.

A similar design could be used to test different concentrations of menthol, for example, instead of 0.5% menthol with varying concentrations of β-alanine as described above, a set concentration of β-alanine (e.g., 5% w/w) with varying concentrations of menthol (e.g., 0, 0.25%, 0.5%, 1%, 1.5%, or 2% w/w) can be tested in a similar manner. In addition, other TRPM8 agonists can be substituted for menthol and tested similarly as described here.

It will be apparent that the precise details of the methods or compositions described may be varied or modified without departing from the spirit of the described aspects of the disclosure. We claim all such modifications and variations that fall within the scope and spirit of the claims below.

1. A topical composition comprising an effective amount of β-alanine and an effective amount of a transient receptor potential cation channel subfamily M (melastatin) member 8 (TRPM8) agonist.

2. The topical composition of claim 1, wherein the TRPM8 agonist is menthol, icilin, crysim-1, borneol, linalool, geraniol, hydroxy-citronellal, WS-12, Frescolat MGA, Frescolat ML, PMD 38, Coolact P, M8-Ag, Cooling Agent 10, or a combination thereof.

3. The topical composition of claim 1, wherein the TRPM8 agonist is menthol.

4. The topical composition of claim 1, comprising:
a) about 0.2% to about 15% weight by weight (w/w) β-alanine;
b) about 1% to about 10% w/w β-alanine; or
c) about 2.5% to about 7.5% w/w β-alanine.

5. The topical composition of claim 1, comprising about 5% w/w β-alanine.

6. The topical composition of claim 3, comprising:
a) about 0.1% to about 5% w/w menthol;
b) about 0.1% to about 2% w/w menthol; or
c) about 0.5% to about 1.5% w/w menthol.

7. The topical composition of claim 3, comprising about 0.5% w/w menthol.

8. The topical composition of claim 3, comprising about 5% w/w β-alanine and about 0.5% w/w menthol.

9. The topical composition of claim 1, further comprising a topical ultraviolet light protectant (UV protectant).

10. The topical composition of claim 9, wherein the topical UV protectant comprises aminobenzoic acid, avobenzone, cinoxate, dioxybenzone, drometrizole trisiloxane, homosalate, meradimate, octocrylene, octinoxate, octisalate, oxybenzone, padimate O, para-aminobenzoic acid (PABA), ensulizole, sulisobenzone, titanium dioxide, trolamine salicylate, zinc oxide, ethylhexyl triazone, ecamsule, silatriazole, bemotrizinol, bisoctrizole, iron oxide, red veterinary petrolatum, kaolin clay, or combinations thereof.

11. The topical composition of claim 1, further comprising a topical antibiotic, antiparasitic, steroid, non-steroidal anti-inflammatory agent, calcineurin inhibitor, JAK inhibitor, analgesic, α2-adrenergic receptor agonist, α1A-adrenoceptor agonist, or combinations thereof.

12. The topical composition of claim 1, further comprising metronidazole, clindamycin, ketoconazole, itraconazole, clotrimazole, voriconazole, posaconazole, miconazole, a corticosteroid, azelaic acid, non-steroidal anti-inflammatory drug (NSAID), crisaborole, ivermectin, tacrolimus, pimecrolimus, ruxolitinib, brimonidine, oxymetazoline, or combinations thereof.

13. The topical composition of claim 1, wherein the topical composition does not comprise an anesthetic other than a TRPM8 agonist or a topical steroid.

14. The topical composition of claim 1, wherein the topical composition does not comprise camphor or lidocaine.

15. The topical composition of claim 1, wherein the topical composition is formulated as an ointment, cream, gel, or lotion.

16. The topical composition of claim 15, wherein the composition is formulated as and emollient or moisturizer.

17. The topical composition of claim 1, formulated for facial application.

18. The topical composition of claim 1, formulated as a shampoo or soap.

19-24. (canceled)

25. A method of decreasing frequency or duration of rosacea symptoms in a subject, comprising:
selecting a subject with rosacea; and
administering a topical composition to an area of skin affected by rosacea, wherein the topical composition comprises β-alanine and a transient receptor potential cation channel subfamily M (melastatin) member 8 (TRPM8) agonist, thereby decreasing the frequency or duration of rosacea symptoms in the subject.

26-29. (canceled)

30. A method of decreasing skin inflammation or redness in a subject, comprising:
selecting a subject with skin inflammation or redness; and
administering an effective amount of a topical composition to an area of skin affected by inflammation or redness, wherein the topical composition comprises β-alanine and a transient receptor potential cation channel subfamily M (melastatin) member 8 (TRPM8) agonist,
thereby decreasing skin inflammation or redness.

31. The method of claim 30, wherein the skin inflammation or redness is caused by rosacea, acute or chronic urticaria, dermatographia, mastocytosis, dermatitis, acne, psoriasis, scleroderma, a wound, or chronic skin flushing, optionally wherein the chronic skin flushing is induced by exercise, diet, or emotion.

32. The method of claim 30, wherein the TRPM8 agonist is menthol.

33. The method of claim 31, wherein the topical composition comprises:
a) about 1% to about 15% weight by weight (w/w) β-alanine;
b) about 2.5% to about 10% w/w β-alanine; or
c) about 2.5% to about 7.5% w/w β-alanine.

34. (canceled)

35. The method of claim 32, wherein the topical composition comprises:
a) about 0.1% to about 5% w/w menthol;
b) about 0.1% to about 2% w/w menthol; or
c) about 0.5% to about 1.5% w/w menthol.

36. (canceled)

37. The method of claim 32, wherein the topical composition comprises about 5% w/w β-alanine and about 0.5% w/w menthol.

38. The method of claim 30, wherein the topical composition is applied to a facial area of the subject.
